(19) United States
(12) Patent Application Publication
(10) Pub. No.: US 2023/0233108 A1
(43) Pub. Date: Jul. 27, 2023

(54) COMBINED INVASIVE AND NON-INVASIVE SENSING

(71) Applicant: Know Labs, Inc., Seattle, WA (US)

(72) Inventor: Phillip BOSUA, Seattle, WA (US)

(21) Appl. No.: 17/584,845

(22) Filed: Jan. 26, 2022

(51) Int. Cl. A61B 5/145; A61B 5/00; A61B 5/1495

(52) U.S. Cl. CPC A61B 5/14503; A61B 5/0004; A61B 5/0031; A61B 5/0022; A61B 5/14532; A61B 5/4848; A61B 5/1495

(57) ABSTRACT

Systems include an invasive sensor and a non-invasive sensor for detection of analytes. The invasive sensor detects one or more non-invasively detected analytes, and the non-invasive sensor detects one or more invasively detected analytes. The one or more non-invasively detected analytes and the one or more invasively detected analytes can include at least one analyte in common, or do not include any analytes in common. The detection of the one or more non-invasively detected analytes and the detection of the one or more invasively detected analytes can be used to

### FIELD

This disclosure is directed to combinations of invasive and non-invasive sensing for detection of one or more analytes in a subject.

### BACKGROUND

There is interest in being able to detect and/or measure an analyte within a target. One example is measuring glucose in biological tissue. In the example of measuring glucose in a patient, current analyte measurement methods are invasive in that they perform the measurement on a bodily fluid such as blood for fingerstick or laboratory-based tests, or on fluid that is drawn from the patient often using an invasive transcutaneous device. There are non-invasive methods that claim to be able to perform glucose measurements in biological tissues. However, many of the non-invasive methods generally suffer from: lack of specificity to the analyte of interest, such as glucose; interference from temperature fluctuations; interference from skin compounds (i.e. sweat) and pigments; and complexity of placement, i.e. the sensing device resides on multiple locations on the patient's body. Further, non-invasive measurements may be limited in the ability to measure certain analytes and/or be used for diagnosis of particular conditions.

### SUMMARY

This disclosure is directed to combinations of invasive and non-invasive sensing for detection of one or more analytes in a subject.

By combining the detection of one or more analytes using a non-invasive sensor with detection of another one or more analytes using an invasive sensor, calibration can be performed and/or combinations of the measured analytes can be used to determine particular conditions in a subject. The combination of invasive and non-invasive measurement can allow for calibration of one of the sensors based on the other of the sensors, for example calibrating the non-invasive sensor for more reliable or accurate future measurements. The combination of invasive and non-invasive measurement can allow a multi-factor analysis of a condition, for example to make a prediction or a diagnosis of a condition based on different measurements respectively obtained by the invasive and non-invasive sensors.

In an embodiment, a measurement system includes a non-invasive sensor configured to detect one or more non-invasively detected analytes in a subject. The non-invasive sensor includes at least one transmit antenna positioned and arranged to transmit a transmit signal into a target containing the one or more non-invasively detected analytes and a transmit circuit that is electrically connectable to the at least one transmit antenna and configured to generate a transmit signal to be transmitted by the at least one transmit antenna. The transmit signal is in a radio or microwave frequency range of the electromagnetic spectrum. The non-invasive sensor further includes at least one receive antenna, positioned and arranged to detect a response resulting from transmission of the transmit signal by the at least one transmit antenna into the target containing the one or more non-invasively detected analytes. The non-invasive sensor further includes a receive circuit that is electrically connectable to the at least one receive antenna, the receive circuit is configured to receive a response detected by the at least one receive antenna. The system further includes an invasive sensor configured to detect one or more invasively detected analytes in the subject and a processor configured to receive data from the invasive sensor and to receive data from the non-invasive sensor.

In an embodiment, the processor is configured to determine a calibration factor for the non-invasive sensor by processing the data from the invasive sensor and the data from the non-invasive sensor. In an embodiment, the processor is configured to assess a condition by processing the data from the invasive sensor and the data from the non-invasive sensor.

In an embodiment, the one or more non-invasively detected analytes and the one or more invasively detected analytes include at least one analyte in common. In an embodiment, the one or more non-invasively detected analytes and the one or more invasively detected analytes do not include any analytes in common. In an embodiment, the invasive sensor is a glucose sensor. In an embodiment, the invasive sensor is an implanted sensor. In an embodiment, the invasive sensor measures a sample taken from the subject. In an embodiment, the sample is a tissue sample. In an embodiment, the sample is a fluid sample.

In an embodiment, the processor is housed in a device including one of the invasive sensor or the non-invasive sensor. In an embodiment, the processor is housed in a device separate from the invasive sensor and the non-invasive sensor. In an embodiment, the processor is included in a cloud server.

In an embodiment, a method of operating a plurality of sensors includes obtaining data regarding detection of one or more non-invasively detected analytes in a subject from a non-invasive sensor. The non-invasive sensor detects the one or more non-invasively detected analytes by a method including generating a transmit signal having at least two different frequencies each of which falls within a range of between about 10 kHz to about 100 GHz, transmitting the transmit signal into a target containing the one or more non-invasively detected analytes from at least one transmit element, and using at least one receive element to detect a response resulting from transmitting the transmit signal by the at least one transmit element into the target containing the one or more non-invasively detected analytes. The method of operating the plurality of sensors further recites obtaining data regarding detection of one or more invasively detected analytes in the subject from an invasive sensor. The method of operating the plurality of sensors further includes receiving the data regarding the detection of the one or more non-invasively detected analytes at a processor and receiving the data regarding the detection of the one or more invasively detected analytes at the processor.

In an embodiment, the method of operating the plurality of sensors further includes processing the data regarding the detection of the one or more non-invasively detected analytes and the data regarding the detection of the one or more invasively detected analytes to determine a calibration factor for the non-invasive sensor and calibrating the non-invasive sensor based on the calibration factor. In an embodiment, the method of operating the plurality of sensors further includes processing the data regarding the detection of the one or more non-invasively detected analytes and the data regarding the detection of the one or more invasively detected analytes to determine a calibration factor for the invasive sensor and calibrating the invasive sensor based on the calibration factor. In an embodiment, the one or more non-invasively detected analytes and the one or more invasively detected analytes include at least one analyte in common. In an embodiment, the one or more non-invasively detected analytes and the one or more invasively detected analytes do not include any analytes in common. In an embodiment, the method of operating the plurality of sensors includes processing the data regarding the detection of the one or more non-invasively detected analytes and the data regarding the detection of the one or more invasively detected analytes to obtain a plurality of assessment factors, and determining an assessment of a condition. In an embodiment, the condition is a response to a treatment.

### DRAWINGS

FIG. 1A shows a sensor system according to an embodiment.

FIG. 1B shows a sensor system according to another embodiment.

FIG. 1C shows a sensor system according to another embodiment.

FIG. 2 shows a method of sensing according to an embodiment.

FIG. 3 shows a method of calibrating a sensor according to an embodiment.

FIG. 4 shows a method of multifactor assessment of a condition according to an embodiment.

### DETAILED DESCRIPTION

This disclosure is directed to combinations of invasive and non-invasive sensing for detection of one or more analytes in a subject.

The transmit antenna and the receive antenna can be located near the target and operated as further described herein to assist in detecting at least one analyte in the target. The transmit antenna transmits a signal, which has at least two frequencies in the radio or microwave frequency range, toward and into the target. The signal with the at least two frequencies can be formed by separate signal portions, each having a discrete frequency, that are transmitted separately at separate times at each frequency. In another embodiment, the signal with the at least two frequencies may be part of a complex signal that includes a plurality of frequencies including the at least two frequencies. The complex signal can be generated by blending or multiplexing multiple signals together followed by transmitting the complex signal whereby the plurality of frequencies are transmitted at the same time. One possible technique for generating the complex signal includes, but is not limited to, using an inverse Fourier transformation technique. The receive antenna detects a response resulting from transmission of the signal by the transmit antenna into the target containing the at least one analyte of interest.

The transmit antenna and the receive antenna are decoupled (which may also be referred to as detuned or the like) from one another. Decoupling refers to intentionally fabricating the configuration and/or arrangement of the transmit antenna and the receive antenna to minimize direct communication between the transmit antenna and the receive antenna, preferably absent shielding. Shielding between the transmit antenna and the receive antenna can be utilized. However, the transmit antenna and the receive antenna are decoupled even without the presence of shielding.

The signal(s) detected by the receive antenna can be analyzed to detect the analyte based on the intensity of the received signal(s) and reductions in intensity at one or more frequencies where the analyte absorbs the transmitted signal. Examples of detecting an analyte using a non-invasive spectroscopy sensor operating in the radio or microwave frequency range of the electromagnetic spectrum are described in WO 2019/217461, U.S. Pat. Nos. 11,063,373, 11,058,331, and 11,033,208 the entire contents of which are incorporated herein by reference. The signal(s) detected by the receive antenna can be complex signals including a plurality of signal components, each signal component being at a different frequency. In an embodiment, the detected complex signals can be decomposed into the signal components at each of the different frequencies, for example through a Fourier transformation. In an embodiment, the complex signal detected by the receive antenna can be analyzed as a whole (i.e. without demultiplexing the complex signal) to detect the analyte as long as the detected signal provides enough information to make the analyte detection. In addition, the signal(s) detected by the receive antenna can be separate signal portions, each having a discrete frequency.

In one embodiment, the sensor described herein can be used to detect the presence of at least one analyte in a target. In another embodiment, the sensor described herein can detect an amount or a concentration of the at least one analyte in the target. The target can be any target containing at least one analyte of interest that one may wish to detect. The target can be human or non-human, animal or non-animal, biological or non-biological. For example, the target can include, but is not limited to, human tissue, animal tissue, plant tissue, an inanimate object, soil, a fluid, genetic material, or a microbe. Non-limiting examples of targets include, but are not limited to, a fluid, for example blood, interstitial fluid, cerebral spinal fluid, lymph fluid or urine, human tissue, animal tissue, plant tissue, an inanimate object, soil, genetic material, or a microbe.

The analyte(s) can be any analyte that one may wish to detect. The analyte can be human or non-human, animal or non-animal, biological or non-biological. For example, the analyte(s) can include, but is not limited to, one or more of glucose, alcohol, white blood cells, or luteinizing hormone. The analyte(s) can include, but is not limited to, a chemical, a combination of chemicals, a virus, bacteria, or the like. The analyte can be a chemical included in another medium, with non-limiting examples of such media including a fluid containing the at least one analyte, for example blood, interstitial fluid, cerebral spinal fluid, lymph fluid or urine, human tissue, animal tissue, plant tissue, an inanimate object, soil, genetic material, or a microbe. The analyte(s) may also be a non-human, non-biological particle such as a mineral or a contaminant.

The analyte(s) can include, for example, naturally occurring substances, artificial substances, metabolites, and/or reaction products. As non-limiting examples, the at least one analyte can include, but is not limited to, insulin, acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); biotinidase; biopterin; c-reactive protein; carnitine; pro-BNP; BNP; troponin; carnosinase; CD4; ceruloplasmin; chenodeoxycholic acid; chloroquine; cholesterol; cholinesterase; conjugated 1-β hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, Duchenne/Becker muscular dystrophy, analyte-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free (3-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; analyte-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, β); lysozyme; mefloquine; netilmicin; phenobarbitone; phenytoin; phytanic/pristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprae, Mycoplasma pneumoniae, Myoglobin, Onchocerca volvulus, parainfluenza virus, Plasmodium falciparum, polio virus, Pseudomonas aeruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin.

The analyte(s) can also include one or more chemicals introduced into the target. The analyte(s) can include a marker such as a contrast agent, a radioisotope, or other chemical agent. The analyte(s) can include a fluorocarbon-based synthetic blood. The analyte(s) can include a drug or pharmaceutical composition, with non-limiting examples including ethanol or other alcohols; ketones; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbiturates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The analyte(s) can include other drugs or pharmaceutical compositions. The analyte(s) can include neurochemicals or other chemicals generated within the body, such as, for example, ascorbic acid, uric acid, dopamine, noradrenaline, 3-methoxytyramine (3MT), 3,4-Dihydroxyphenylacetic acid (DOPAC), Homovanillic acid (HVA), 5-Hydroxytryptamine (5HT), and 5-Hydroxyindoleacetic acid (FHIAA).

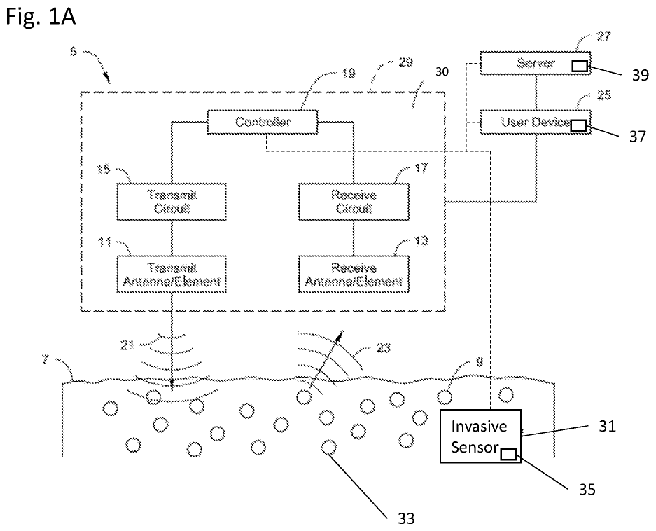

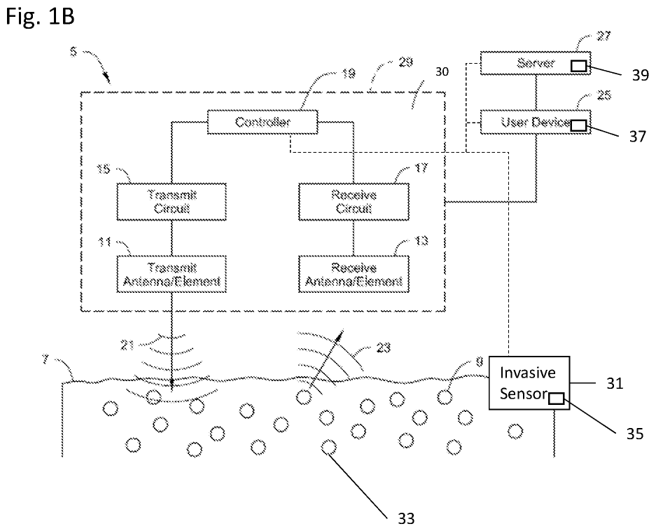

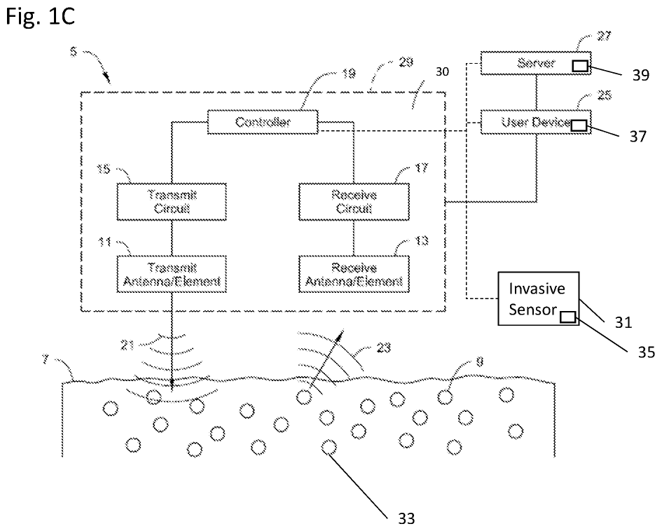

Referring now to FIGS. 1A-1C, each shows an embodiment of an analyte sensor system 3 with a non-invasive analyte sensor 5 and an invasive sensor 31. The non-invasive analyte sensor 5 is depicted relative to a target 7 that contains one or more non-invasively detected analytes of interest 9. In this example, the non-invasive analyte sensor 5 is depicted as including an antenna array that includes a transmit antenna/element 11 (hereinafter “transmit antenna 11”) and a receive antenna/element 13 (hereinafter “receive antenna 13”). The sensor 5 further includes a transmit circuit 15, a receive circuit 17, and a controller 19. The non-invasive analyte sensor 5 is configured to detect the one or more non-invasively detected analytes of interest 9 in the target 7 without physical disruption to the target, as described below using transmission of electromagnetic signals and detection of responses for detection of the one or more non-invasively detected analytes of interest 9. Target 7 can be a living subject, for example a person in which the one or more analytes of interest 9 are to be detected. Target 7 can be a portion of a living subject, such as skin, blood, interstitial fluid, or the like.

The transmit antenna 11 is positioned, arranged and configured to transmit a signal 21 that is the radio frequency (RF) or microwave range of the electromagnetic spectrum into the target 7. The transmit antenna 11 can be an electrode or any other suitable transmitter of electromagnetic signals in the radio frequency (RF) or microwave range. The transmit antenna 11 can have any arrangement and orientation relative to the target 7 that is sufficient to allow the analyte sensing to take place. In one non-limiting embodiment, the transmit antenna 11 can be arranged to face in a direction that is substantially toward the target 7.

The signal 21 transmitted by the transmit antenna 11 is generated by the transmit circuit 15 which is electrically connectable to the transmit antenna 11. The transmit circuit 15 can have any configuration that is suitable to generate a transmit signal to be transmitted by the transmit antenna 11. Transmit circuits for generating transmit signals in the RF or microwave frequency range are well known in the art. In one embodiment, the transmit circuit 15 can include, for example, a connection to a power source, a frequency generator, and optionally filters, amplifiers or any other suitable elements for a circuit generating an RF or microwave frequency electromagnetic signal. In an embodiment, the signal generated by the transmit circuit 15 can have at least two discrete frequencies (i.e. a plurality of discrete frequencies), each of which is in the range from about 10 kHz to about 100 GHz. In another embodiment, each of the at least two discrete frequencies can be in a range from about 300 MHz to about 6000 MHz. In an embodiment, the transmit circuit 15 can be configured to sweep through a range of frequencies that are within the range of about 10 kHz to about 100 GHz, or in another embodiment a range of about 300 MHz to about 6000 MHz. In an embodiment, the transmit circuit 15 can be configured to produce a complex transmit signal, the complex signal including a plurality of signal components, each of the signal components having a different frequency. The complex signal can be generated by blending or multiplexing multiple signals together followed by transmitting the complex signal whereby the plurality of frequencies are transmitted at the same time.

The receive antenna 13 is positioned, arranged, and configured to detect one or more electromagnetic response signals 23 that result from the transmission of the transmit signal 21 by the transmit antenna 11 into the target 7 and impinging on the one or more non-invasively detected analyte(s) 9. The receive antenna 13 can be an electrode or any other suitable receiver of electromagnetic signals in the radio frequency (RF) or microwave range. In an embodiment, the receive antenna 13 is configured to detect electromagnetic signals having at least two frequencies, each of which is in the range from about 10 kHz to about 100 GHz, or in another embodiment a range from about 300 MHz to about 6000 MHz. The receive antenna 13 can have any arrangement and orientation relative to the target 7 that is sufficient to allow detection of the response signal(s) 23 to allow the analyte sensing to take place. In one non-limiting embodiment, the receive antenna 13 can be arranged to face in a direction that is substantially toward the target 7.

The receive circuit 17 is electrically connectable to the receive antenna 13 and conveys the received response from the receive antenna 13 to the controller 19. The receive circuit 17 can have any configuration that is suitable for interfacing with the receive antenna 13 to convert the electromagnetic energy detected by the receive antenna 13 into one or more signals reflective of the response signal(s) 23. The construction of receive circuits are well known in the art. The receive circuit 17 can be configured to condition the signal(s) prior to providing the signal(s) to the controller 19, for example through amplifying the signal(s), filtering the signal(s), or the like. Accordingly, the receive circuit 17 may include filters, amplifiers, or any other suitable components for conditioning the signal(s) provided to the controller 19. In an embodiment, at least one of the receive circuit 17 or the controller 19 can be configured to decompose or demultiplex a complex signal, detected by the receive antenna 13, including a plurality of signal components each at different frequencies into each of the constituent signal components. In an embodiment, decomposing the complex signal can include applying a Fourier transform to the detected complex signal. However, decomposing or demultiplexing a received complex signal is optional. Instead, in an embodiment, the complex signal detected by the receive antenna can be analyzed as a whole (i.e. without demultiplexing the complex signal) to detect the analyte as long as the detected signal provides enough information to make the analyte detection.

The controller 19 controls the operation of the sensor 5. The controller 19, for example, can direct the transmit circuit 15 to generate a transmit signal to be transmitted by the transmit antenna 11. The controller 19 further receives signals from the receive circuit 17. The controller 19 can optionally process the signals from the receive circuit 17 to detect the analyte(s) 9 in the target 7. In one embodiment, the controller 19 may optionally be in communication with at least one external device 25 such as a user device and/or a remote server 27, for example through one or more wireless connections such as Bluetooth, wireless data connections such a 4G, 5G, LTE or the like, or Wi-Fi. If provided, the external device 25 and/or remote server 27 may process (or further process) the signals that the controller 19 receives from the receive circuit 17, for example to detect the one or more non-invasively detected analyte(s) 9. If provided, the external device 25 may be used to provide communication between the sensor 5 and the remote server 27, for example using a wired data connection or via a wireless data connection or Wi-Fi of the external device 25 to provide the connection to the remote server 27. In an embodiment, the controller 19 is further configured to can process the data from non-invasive sensor 5 along with data from the invasive sensor 31. In an embodiment, another controller separate from controller 19 can process the data from non-invasive sensor 5 along with data from the invasive sensor 31. This additional controller can be included in, for example, external device 25 or remote server 27.

With continued reference to FIGS. 1A-1C, the sensor 5 may include a sensor housing 29 (shown in dashed lines) that defines an interior space 30. Components of the sensor 5 may be attached to and/or disposed within the housing 29. For example, the transmit antenna 11 and the receive antenna 13 are attached to the housing 29. In some embodiments, the antennas 11, 13 may be entirely or partially within the interior space 30 of the housing 29. In some embodiments, the antennas 11, 13 may be attached to the housing 29 but at least partially or fully located outside the interior space 30. In some embodiments, the transmit circuit 15, the receive circuit 17 and the controller 19 are attached to the housing 29 and disposed entirely within the sensor housing 29.

System 3 further includes invasive sensor 31. Invasive sensor 31 is a sensor configured to detect one or more invasively detected analytes of interest 33 within the subject including target 7. The invasive sensor is configured such that there is disruption to tissue of the subject including target 7 as part of obtaining measurements from invasive sensor 31, for example through obtaining a tissue sample, being implanted into the subject, requiring a withdrawal of a fluid sample such as blood through a finger stick or other such method, or the like. In an embodiment, invasive sensor 31, the one or more invasively detected analytes of interest 33 and the one or more non-invasively detected analytes of interest 9 include at least one analyte in common. In an embodiment, the one or more invasively detected analytes of interest 33 and the one or more non-invasively detected analytes of interest 9 are identical with respect to the included analytes. In an embodiment, the one or more non-invasively detected analytes of interest 9 and the one or more invasively detected analytes of interest 33 do not include any analytes in common with one another. In an embodiment, the invasive sensor 31 is an implantable sensor, for example as shown in FIG. 1A. In an embodiment, the invasive sensor 31 can reside on or under a subject's skin during use, for example as shown in FIG. 1B. Non-limiting examples of invasive sensors include glucose sensors such as the Dexcom® G6 CGM or the Freestyle Libre™. In an embodiment, the invasive sensor 31 is an implantable glucose sensor. In an embodiment, the invasive sensor 31 detects the analyte based on a sample obtained from the subject including target 7. In an embodiment, the invasive sensor can be separate from the subject but still sense in an invasive manner, for example as shown in FIG. 1C. For example, the invasive sensor 31 can be configured to receive a sample such as a fluid sample and/or a tissue sample obtained from the subject and analyze said fluid sample and/or tissue sample to detect the one or more invasively detected analytes of interest 33. In an embodiment, the invasive sensor 31 can be a glucose sensor configured to measure glucose based on a blood sample, such as a finger-stick glucose sensor. Non-limiting examples of invasive glucose sensors include the Dexcom® G6 CGM or the Freestyle Libre™ glucose sensors.

Invasive sensor 31 can be configured to communicate with one or more of the non-invasive sensor 5, the external device 25, and/or remote server 27. The communication can be any suitable communication of signals to and/or from the invasive sensor 31, non-invasive sensor 5, external device 25, and/or remote server 27, such as wired or wireless communications. In an embodiment, invasive sensor 31 includes a processor 35 configured to receive data from sensor 5 and process the data obtained at invasive sensor 31 and the data received from non-invasive sensor 5. In an embodiment, external device 25 includes a processor 37 configured to receive data from sensor 5 and data from invasive sensor 31 and to process the data received from the non-invasive sensor 5 and the invasive sensor 31. In an embodiment, external device 25 includes a processor 37 configured to receive data from sensor 5 and data from invasive sensor 31 and to process the data received from the non-invasive sensor 5 and the invasive sensor 31. In an embodiment, remote server 27 includes a processor 39 configured to receive data from sensor 5 and data from invasive sensor 31 and to process the data received from the non-invasive sensor 5 and the invasive sensor 31. In embodiments, two or more of the processors 35, 37, and 39 can communicate with one another and process data from sensor 5 and from invasive sensor 31 together, for example in parallel or in performing different processing steps on said data. In embodiments, non-invasive sensor 5 and invasive sensor 31 can be operated at times that overlap. In an embodiment, non-invasive sensor 5 is operated prior to operation of invasive sensor 31. In an embodiment, invasive sensor 5 is operated prior to operation of invasive sensor 31. The times of operation can be according to any suitable protocol, for example defined schedules for operation of each of non-invasive sensor 5 and invasive sensor 31, defined timing for operation of one of non-invasive sensor 5 or invasive sensor 31 based on operation of the other, or the like.

The receive antenna 13 can be decoupled or detuned with respect to the transmit antenna 11 such that electromagnetic coupling between the transmit antenna 11 and the receive antenna 13 is reduced. The decoupling of the transmit antenna 11 and the receive antenna 13 increases the portion of the signal(s) detected by the receive antenna 13 that is the response signal(s) 23 from the target 7, and minimizes direct receipt of the transmitted signal 21 by the receive antenna 13. The decoupling of the transmit antenna 11 and the receive antenna 13 results in transmission from the transmit antenna 11 to the receive antenna 13 having a reduced forward gain and an increased reflection at output compared to antenna systems having coupled transmit and receive antennas. In an embodiment, the transmit antenna 11 and/or the receive antenna 13 can be shape-changing antennas such as arrays of controllable circuits, controllable conductive materials, or the like. When used as transmit antenna 11 and/or receive antenna 13, the shape-changing antennas can be formed at specific times so as to reduce or eliminate direct receipt of transmitted signal 21 at receive antenna 13. When used as transmit antenna and/or receive antenna 13 can have shapes and/or positions selected such that transmit antenna 11 and receive antenna 13 are decoupled from one another.

In an embodiment, coupling between the transmit antenna 11 and the receive antenna 13 is 95% or less. In another embodiment, coupling between the transmit antenna 11 and the receive antenna 13 is 90% or less. In another embodiment, coupling between the transmit antenna 11 and the receive antenna 13 is 85% or less. In another embodiment, coupling between the transmit antenna 11 and the receive antenna 13 is 75% or less.

Any technique for reducing coupling between the transmit antenna 11 and the receive antenna 13 can be used. For example, the decoupling between the transmit antenna 11 and the receive antenna 13 can be achieved by one or more intentionally fabricated configurations and/or arrangements between the transmit antenna 11 and the receive antenna 13 that is sufficient to decouple the transmit antenna 11 and the receive antenna 13 from one another.

For example, the decoupling of the transmit antenna 11 and the receive antenna 13 can be achieved by intentionally configuring the transmit antenna 11 and the receive antenna 13 to have different geometries from one another. Intentionally different geometries refers to different geometric configurations of the transmit and receive antennas 11, 13 that are intentional. Intentional differences in geometry are distinct from differences in geometry of transmit and receive antennas that may occur by accident or unintentionally, for example due to manufacturing errors or tolerances.

Another technique to achieve decoupling of the transmit antenna 11 and the receive antenna 13 is to provide appropriate spacing between each antenna 11, 13 that is sufficient to decouple the antennas 11, 13 and force a proportion of the electromagnetic lines of force of the transmitted signal 21 into the target 7 thereby minimizing or eliminating as much as possible direct receipt of electromagnetic energy by the receive antenna 13 directly from the transmit antenna 11 without traveling into the target 7. The appropriate spacing between each antenna 11, 13 can be determined based upon factors that include, but are not limited to, the output power of the signal from the transmit antenna 11, the size of the antennas 11, 13, the frequency or frequencies of the transmitted signal, and the presence of any shielding between the antennas. This technique helps to ensure that the response detected by the receive antenna 13 is measuring the analyte 9 and is not just the transmitted signal 21 flowing directly from the transmit antenna 11 to the receive antenna 13. In some embodiments, the appropriate spacing between the antennas 11, 13 can be used together with the intentional difference in geometries of the antennas 11, 13 to achieve decoupling.

In one embodiment, the transmit signal that is transmitted by the transmit antenna 11 can have at least two different frequencies, for example upwards of 7 to 12 different and discrete frequencies. In another embodiment, the transmit signal can be a series of discrete, separate signals with each separate signal having a single frequency or multiple different frequencies.

In one embodiment, the transmit signal (or each of the transmit signals) can be transmitted over a transmit time that is less than, equal to, or greater than about 300 ms. In another embodiment, the transmit time can be than, equal to, or greater than about 200 ms. In still another embodiment, the transmit time can be less than, equal to, or greater than about 30 ms. The transmit time could also have a magnitude that is measured in seconds, for example 1 second, 5 seconds, 10 seconds, or more. In an embodiment, the same transmit signal can be transmitted multiple times, and then the transmit time can be averaged. In another embodiment, the transmit signal (or each of the transmit signals) can be transmitted with a duty cycle that is less than or equal to about 50%. In an embodiment, the transmit signal can include frequency sweeps having frequency steps with selected operations times to facilitate comparison of frequency sweep results, as discussed in U.S. Pat. No. 11,033,208, which is herein incorporated by reference in its entirety.

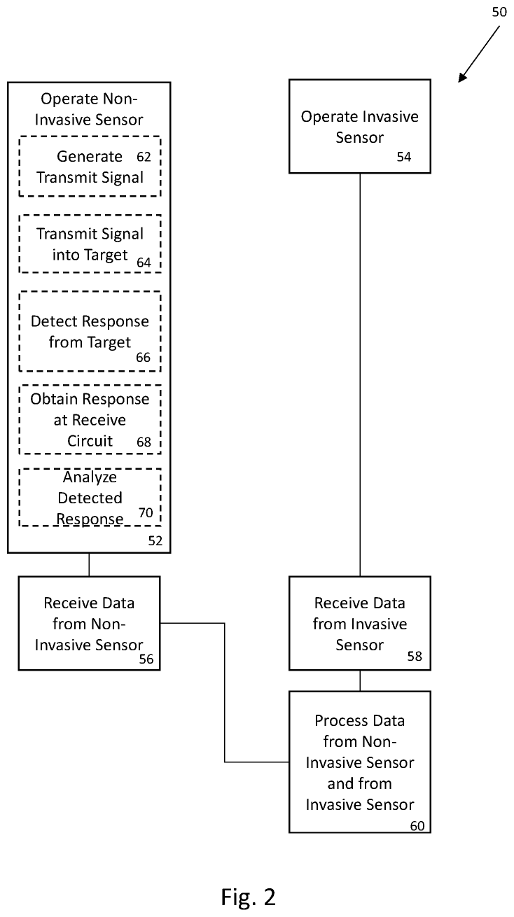

FIG. 2 shows a method of operating sensors according to an embodiment. Method 50 includes operating a non-invasive sensor 52, operating an invasive sensor 54, receiving data from the non-invasive sensor 56, receiving data from the invasive sensor 58, and processing the data received from the non-invasive sensor together with the data received from the invasive sensor 60.

Operating the non-invasive sensor 52 includes detecting one or more non-invasively detected analytes in a subject. The measurement can be an in vivo measurement of the one or more non-invasively detected analytes in the subject. Operating the non-invasive sensor can include generating a transmit signal 62, transmitting the transmit signal into the target 64, detecting a response resulting from the transmit signal interacting with the one or more non-invasively detected analytes of interest 66, and obtaining the detected response at a receive circuit at 68. Optionally, operating the non-invasive sensor 52 can further include analyzed the detected response at 70. The transmit signal can be generated by a transmit circuit such as transmit circuit 15 as discussed above and shown in FIGS. 1A-1C. The transmit signal generated at 62 can include at least two discrete frequencies (i.e. a plurality of discrete frequencies), each of which is in the range from about 10 kHz to about 100 GHz. In another embodiment, each of the at least two discrete frequencies can be in a range from about 300 MHz to about 6000 MHz. In an embodiment, the transmit signal generated at 62 can be a sweep through a range of frequencies. The signal is transmitted into the target by way of a transmit antenna, for example, transmit antenna 11 as discussed above and shown in FIGS. 1A-1C. A response resulting from the transmit signal interacting with the one or more first analytes of interest is detected at 66 by a receive antenna, such as receive antenna 13 as discussed above and shown in FIGS. 1A-1C. The detected response is obtained by a receive circuit such as receive circuit 17 discussed above and shown in FIGS. 1A-1C. In an embodiment, the receive circuit can provide the detected response to a processor, for example for processing of the data from the non-invasive sensor and data from the invasive sensor at 60, or for analysis of the detected response at 70. The optional analysis of the detected response at 70 can include, for example, conditioning of the signal such as, as non-limiting examples, removal of outliers, selection of specific portions of the response such as frequency ranges, demultiplexing of signal components, or the like, or determination of the presence and/or amount of at least some of the one or more non-invasively detected analytes. In an embodiment, the optional analysis of the detected response 70 is performed at the non-invasive sensor, such as at controller 19 discussed above and shown in FIGS. 1A-1C. In an embodiment, optional analysis of the detected response can be performed at one or more of an external device 25 or a remote server 27. In an embodiment, the results of the analysis of the detected signal at 70 can be the data from the non-invasive sensor that is processed with the data from the invasive sensor at 60.

Operating the invasive sensor 54 includes detecting one or more invasively detected analytes in the subject. The detection of the one or more invasively detected analytes can be any suitable detections to be performed by the invasive sensor being used, such as detection by an implanted sensor, detection based on a sample of tissue and/or blood, or any other suitable detection performed using the invasive sensor. In an embodiment, the one or more invasively detected analytes can include at least one analyte in common with the one or more non-invasively detected analytes detected by operation of the non-invasive sensor at 52. In an embodiment, the one or more invasively detected analytes and the one or more non-invasively detected analytes do not include any analytes in common. In an embodiment, the one or more invasively detected analytes and the one or more non-invasively detected analytes are entirely the same one or more analytes. In an embodiment, operating the invasive sensor at 54 can be performed prior to operating the non-invasive sensor at 52. In an embodiment, operating the invasive sensor at 54 can be performed subsequent to operating the non-invasive sensor at 52. In an embodiment, operating the invasive sensor at 52 and operating the non-invasive sensor at 54 can overlap in time. In an embodiment, operating the invasive sensor at 52 and operating the non-invasive sensor at 54 can be according to predetermined schedules. In an embodiment, one or both of operating the invasive sensor at 52 and operating the non-invasive sensor at 54 can be based on a user input or a trigger event. In an embodiment, operating the invasive sensor at 52 and operating the non-invasive sensor at 54 can be done in relation to one another, for example by having operation of one of the invasive sensor or the non-invasive sensor trigger operation of the other, or having a delay period between operating one of the invasive sensor and the non-invasive sensor and then operating the other.

Data from the non-invasive sensor is received at 56. The data received from the non-invasive sensor at 56 can be, for example, raw data from the non-invasive sensor such as the signals received at the receive circuit of the sensor or processed data such as data indicative of the presence and/or amount of the one or more non-invasively detected analytes. The data can be received at a memory for subsequent processing of the data from the non-invasive sensor and data from the invasive sensor are processed at 60. The data can be received at a processor where the data from the non-invasive sensor and data from the invasive sensor are processed at 60. The memory or processor receiving the data at 56 can be included in the non-invasive sensor, included in the invasive sensor, included in an external device, or located at a remote server. The data can be received by way of any suitable form of communication, such as wired or wireless communications from the non-invasive sensor to the memory or processor where the data from the non-invasive sensor is received at 56.

Data from the invasive sensor is received at 58. The data received from the invasive sensor at 58 can be, for example, raw data from the non-invasive sensor such as the signals received at the receive circuit of the sensor or processed data such as data indicative of the presence and/or amount of the one or more invasively detected analytes. The data can be received at a memory for subsequent processing of the data from the invasive sensor and data from the non-invasive sensor are processed at 60. The data can be received at a processor where the data from the non-invasive sensor and data from the invasive sensor are processed at 60. The memory or processor receiving the data at 58 can be included in the invasive sensor, included in the non-invasive sensor, included in an external device, or located at a remote server. The data can be received by way of any suitable form of communication, such as wired or wireless communications from the non-invasive sensor to the memory or processor where the data from the invasive sensor is received at 58.

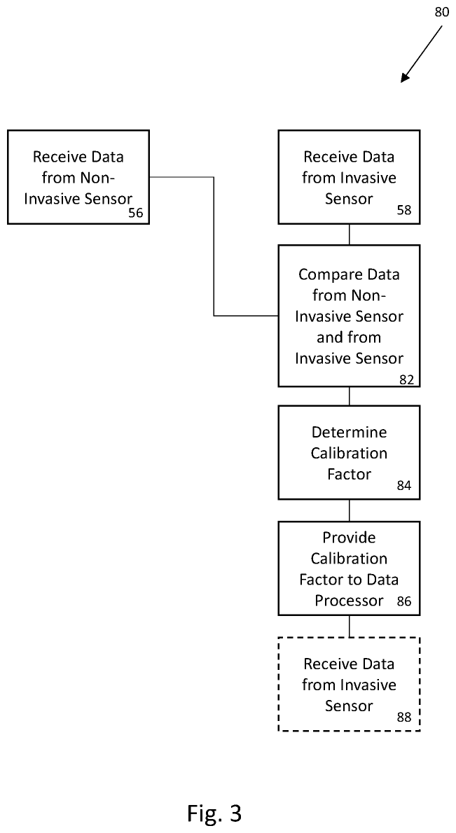

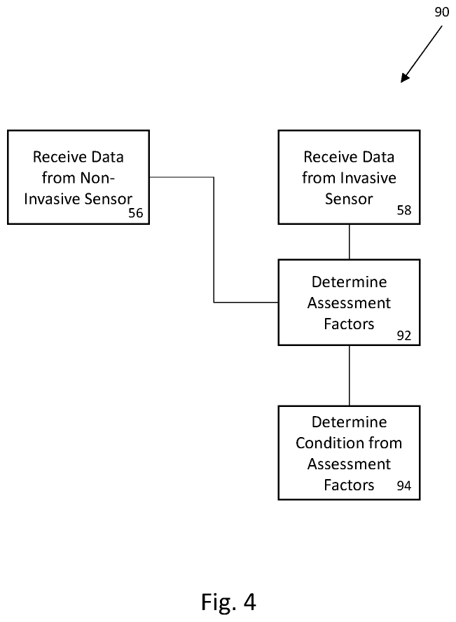

The data received from the non-invasive sensor at 56 and the data received from the invasive sensor at 58 are processed at 60. In an embodiment, the processing of the data from the non-invasive sensor and the data from the invasive sensor at 60 can be used to calibrate the non-invasive sensor. Calibration of the non-invasive sensor is shown in FIG. 3 and discussed below. In an embodiment, the processing of the data from the non-invasive sensor at 60 can be used for providing a multifactor assessment of a condition. Multifactor assessment of a condition by processing the data from the non-invasive sensor and the data from the invasive sensor is shown in FIG. 4 and discussed below. In an embodiment, the processing of the data from the non-invasive sensor and the data from the invasive sensor at 60 can include confirming or validating results from one of the non-invasive sensor or the invasive sensor. The confirmation or validation can be based on, for example, similarity of the results for analytes in common measured by each of the invasive sensor and the non-invasive sensor. In an embodiment, the invasive sensor can be used to confirm or validate results of measurement by the non-invasive sensor. In an embodiment, the non-invasive sensor can be used to confirm or validate results of measurement by the invasive sensor. In an embodiment, the processing of the data from the non-invasive sensor and the data from the invasive sensor at 60 can include determining a measurement value for one or more analytes, such as averaging results of the data from the invasive sensor and the data from the non-invasive sensor. In an embodiment, the averaging can be a weighted average, with greater weight being given to one of the invasive sensor or the non-invasive sensor when averaging the data.

In an embodiment, the processing of the data from the non-invasive sensor and the invasive sensor at 60 can include the determination of a measurement for one or more analytes. The measurement of the one or more analytes can be for one or more analytes in common each measured by the invasive sensor and by the non-invasive sensor. In an embodiment, the measurement can be based on an average of the measurements for the one or more analytes obtained by the invasive sensor and the non-invasive sensor. In an embodiment, the average of the measurements can be a weighted average. Weightings for the average can be based on, for example, accuracy and/or precision of each of the invasive sensor and the non-invasive sensor, such as signal to noise ratios, confidence intervals, error ranges, or the like. In an embodiment, the average can be determined from measurements taken during overlapping periods of time. In an embodiment, the average can be determined from data taken at different times, for example based on differences in measurement between the invasive sensor and the non-invasive sensor, such as differences in the locations where the measurements are taken, differences in times to carry out and/or process results of the measurements, or the like for the invasive sensor and the non-invasive sensor.

FIG. 3 shows a method of calibrating a sensor according to an embodiment. Method 80 includes receiving data from the non-invasive sensor 56 as discussed above, receiving data from the invasive sensor 58 as discussed above. In method 80, the data received from the non-invasive sensor at 56 and the data received from the invasive sensor at 58 can be regarding one or more analytes in common, with at least some of the one or more analytes in common being the at least one analyte to be calibrated for. Method 80 further includes comparing the data from the non-invasive sensor and data from the invasive sensor regarding at least one analyte to be calibrated for 82, determining a calibration factor for one of the non-invasive sensor or the invasive sensor 84, and providing the calibration factor to a data processor for the one of the non-invasive sensor or the invasive sensor 86. In an embodiment, method 80 can further include operating the one of the non-invasive sensor or the invasive sensor using the calibration factor 88.

Data from the non-invasive sensor and data from the invasive sensor regarding at least one analyte to be calibrated for are compared at 82. The comparison can be a comparison of data from each of the non-invasive sensor and the invasive sensor, such as raw data or processed data such as detected amounts of the at least one analyte to be calibrated for. In the comparison, one of the non-invasive sensor or the invasive sensor can be considered the reference sensor, with the other sensor being calibrated. The reference sensor can be the one of the non-invasive sensor or the invasive sensor that is considered to be more reliable, for example the sensor that is already calibrated, the sensor that is calibrated to a more precise and/or accurate standard, the sensor using an inherently more reliable measurement, or the like. In an embodiment, the data from the reference sensor is processed data indicative of the amount or amounts of the at least one analyte to be calibrated for.

A calibration factor for one of the non-invasive sensor or the invasive sensor is determined at 84. Differences between the data from the reference data and the data from the sensor being calibrated from the comparison at 82 can be used to determine one or more calibration factors for calibration of the non-reference sensor. In an embodiment, the comparison can be between data from the reference sensor and processed data from sensor to be calibrated. In this embodiment, the calibration factor can be an adjustment value to be applied to the processed data at the sensor being calibrated, a modification to processing used to obtain the processed data at the sensor being calibrated, or the like. In an embodiment, the comparison can be between raw or processed data from the sensor to be calibrated and the data from the reference sensor. In this embodiment, the calibration factor can be an association between the data from the sensor to be calibrated and amounts of the at least one analyte to be calibrated for as determined according to the reference data. The calibration factor can be, for example, a table of the associations between data values at the sensor to be calibrated with values for the at least one analyte to be calibrated for, a function associating data at the sensor to be calibrated with values for the at least one analyte being calibrated for, or the like.

The calibration factor is provided to a data processor for the one of the non-invasive sensor or the invasive sensor at 86. The data processor for the one of the non-invasive sensor or the invasive sensor is the processor used to process data from the sensor being calibrated in method 80. In an embodiment, the processor is incorporated into the one of the non-invasive sensor, such as non-invasive sensor 5 shown in FIGS. 1A-1C and described above, or the invasive sensor, such as invasive sensor 31 shown in FIGS. 1A-1C and described above. In an embodiment, the processor is included in a device separate from the sensors, such as external device 25 or remote server 27 as discussed above and shown in FIGS. 1A-1C. The calibration factor can be provided to the data processor through any suitable communication, such as wired or wireless communication, retrieval of the calibration factor from a memory or the like.

The one of the non-invasive sensor or the invasive sensor can be operated according to the calibration factor at 88. The one of the non-invasive sensor or the invasive sensor can be operated to obtain data including data regarding the at least one analyte calibrated for in method 80. The data obtained by operation of the one of the non-invasive sensor or the invasive sensor can be processed using the calibration factor to determine the presence and/or amounts of analytes including the at least one analyte calibrated for.

In embodiments, method 80 may be iterated multiple times, for example to calibrate the sensor being calibrated for a plurality of analytes, to iteratively refine the calibration factor, to obtain a database for calibration of the sensor, or the like.

FIG. 4 shows a method of multifactor assessment of a condition according to an embodiment. Method 90 includes receiving data from the non-invasive sensor at 56, receiving data from the invasive sensor at 58, processing the data from the non-invasive sensor and data from the invasive sensor to determine a plurality of assessment factors 92 and using the plurality of assessment factors 92 to determine a condition 94.

A plurality of assessment factors are determined at 92. The assessment factors determined at 92 can include the presence and/or amount of a plurality of analytes of interest. At least some of the plurality of the analytes of interest can be included in one or more non-invasively detected analytes detected using the non-invasive sensor. At least some of the plurality of analytes of interest can be included in one or more invasively detected analytes detected using the invasive sensor. In an embodiment, the one or more non-invasively detected analytes and the one or more invasively detected analytes can include at least one analyte in common. In an embodiment, the one or more non-invasively detected analytes and the one or more invasively detected analytes can include no analytes in common. The assessment factors can be determined at any suitable point in a system carrying out method 90. In an embodiment, the assessment factors can be determined at one or more of the non-invasive sensor 5, the invasive sensor 31, a remote device 25, and/or a remote server such as remote server 27. In an embodiment, the assessment factors are determined based on measurements from the invasive sensor and the non-invasive sensor that are respectively taken at times that are separate from one another. In an embodiment, the assessment factors are determined based on measurements from the invasive sensor and the non-invasive sensor that are respectively taken at times that are overlapping or simultaneous. In an embodiment, the determination of the assessment factors at 92 can include determining a first one or more assessment factors based on data received from the non-invasive sensor at 56, and separately determining a second one or more assessment factors based on data received from the invasive sensor at 58. In an embodiment, the determining of the first one or more assessment factors and the determination of the second one or more assessment factors can be performed at different locations in the system carrying out method 90. In an embodiment, the determining of the first one or more assessment factors and the determination of the second one or more assessment factors can be performed at a common location in the system carrying out method 90.

A condition is determined at 94 based on the plurality of assessment factors. The condition can be, for example, a diagnosis of a health condition in a subject, a determination of completion of a chemical process, or any other suitable condition that can be determined using the plurality of assessment factors. The condition may be determined by any suitable method using each of the plurality of assessment factors. In an embodiment, the plurality of assessment factors are compared to a model associating the condition with the plurality of assessment factors, such as a multi-dimensional table. In an embodiment, a first one or more of the assessment factors can be used to select a model, and the selected model is used to determine the condition based on a second one or more of the assessment factors. In an embodiment, conditional logic is used to associate the plurality of assessment factors with a condition being determined. The condition determined at 94, can be, as non-limiting examples, a health condition such as cancer, an endocrine condition, heart disease, insulin resistance, or the like. In such embodiments, the assessment factors can be a plurality of analytes associated with the health condition such as a plurality of compounds associated with cancer, a plurality of hormones affected by the endocrine condition, or the like. In embodiments, the condition determined at 94 can be responsiveness to a treatment, for example response to administration of a drug. In such embodiment, the assessment factors can include the presence and/or amount of analytes included in or affected by the treatment. For example, when the condition determined at 94 is response to administration of a drug, the assessment factors can include the presence and/or amount of the drug and the presence and/or amount of one or more analytes resulting from or affected by the drug such as metabolites of the drug and/or compounds affected by the drug. In embodiments where the condition determined at 94 is responsiveness to a treatment, the plurality of assessment factors used to determine the condition can include assessment factors determined prior to, during, and/or subsequent to administration of the treatment to the subject.

The examples disclosed in this application are to be considered in all respects as illustrative and not limitative. The scope of the invention is indicated by the appended claims rather than by the foregoing description; and all changes which come within the meaning and range of equivalency of the claims are intended to be embraced therein.

1. An analyte measurement system, comprising:
a non-invasive analyte sensor configured to detect one or more non-invasively detected analytes in a subject, the non-invasive analyte sensor including:
at least one transmit antenna, the at least one transmit antenna is positioned and arranged to transmit a transmit signal into the subject containing the one or more non-invasively detected analytes;
a transmit circuit that is electrically connectable to the at least one transmit antenna, the transmit circuit configured to generate a transmit signal to be transmitted by the at least one transmit antenna, the transmit signal in a radio or microwave frequency range of the electromagnetic spectrum;
at least one receive antenna, the at least one receive antenna positioned and arranged to detect a response resulting from transmission of the transmit signal by the at least one transmit antenna into the target containing the one or more non-invasively detected analytes; and
a receive circuit that is electrically connectable to the at least one receive antenna, the receive circuit is configured to receive a response detected by the at least one receive antenna;
an invasive analyte sensor configured to detect one or more invasively detected analytes in the subject; and
a processor configured to receive data from the invasive analyte sensor and to receive data from the non-invasive analyte sensor.

2. The analyte measurement system of claim 1, wherein the processor is configured to determine a calibration factor for the non-invasive analyte sensor by processing the data from the invasive analyte sensor and the data from the non-invasive analyte sensor.

3. The analyte measurement system of claim 1, wherein the processor is configured to assess a condition of the subject by processing the data from the invasive analyte sensor and the data from the non-invasive analyte sensor.

4. The analyte measurement system of claim 1, wherein the one or more non-invasively detected analytes and the one or more invasively detected analytes include at least one analyte in common.

5. The analyte measurement system of claim 4, wherein the processor is configured to average the data from the invasive analyte sensor and the data from the non-invasive analyte sensor to determine a measurement of at least one analyte of interest, and the at least one analyte in common includes the at least one analyte of interest.

6. The analyte measurement system of claim 1, wherein the one or more non-invasively detected analytes and the one or more invasively detected analytes do not include any analytes in common.

7. The analyte measurement system of claim 1, wherein the invasive analyte sensor is a glucose sensor.

8. The analyte measurement system of claim 1, wherein the invasive analyte sensor is an implanted sensor.

9. The analyte measurement system of claim 1, wherein the invasive analyte sensor measures a sample taken from the subject.

10. The analyte measurement system of claim 1, wherein the processor is housed in a device including one of the invasive analyte sensor or the non-invasive analyte sensor.

11. The analyte measurement system of claim 1, wherein the processor is housed in a device separate from the invasive analyte sensor and the non-invasive analyte sensor.

12. The analyte measurement system of claim 1, wherein the processor is included in a cloud server.

13. A method of operating a plurality of analyte sensors, comprising:
obtaining data regarding detection of one or more non-invasively detected analytes in a subject from a non-invasive analyte sensor; wherein the non-invasive analyte sensor detects the one or more non-invasively detected analytes by a method including:
generating a transmit signal having at least two different frequencies each of which falls within a range of between about 10 kHz to about 100 GHz;
transmitting the transmit signal into a target containing the one or more non-invasively detected analytes from at least one transmit element; and
using at least one receive element to detect a response resulting from transmitting the transmit signal by the at least one transmit element into the target containing the one or more non-invasively detected analytes;
obtaining data regarding detection of one or more invasively detected analytes in the subject from an invasive analyte sensor;
receiving the data regarding the detection of the one or more non-invasively detected analytes at a processor; and
receiving the data regarding the detection of the one or more invasively detected analytes at the processor.

14. The method of claim 13, further comprising processing the data regarding the detection of the one or more non-invasively detected analytes and the data regarding the detection of the one or more invasively detected analytes to determine a calibration factor for the non-invasive analyte sensor and calibrating the non-invasive analyte sensor based on the calibration factor.

15. The method of claim 13, further comprising processing the data regarding the detection of the one or more non-invasively detected analytes and the data regarding the detection of the one or more invasively detected analytes to determine a calibration factor for the invasive analyte sensor and calibrating the invasive analyte sensor based on the calibration factor.

16. The method of claim 13, wherein the one or more non-invasively detected analytes and the one or more invasively detected analytes include at least one analyte in common.

17. The method of claim 16, further comprising processing the data regarding the detection of the one or more non-invasively detected analytes and the data regarding the detection of the one or more invasively detected analytes to determine measurement values for at least one analyte of interest based on an average of the data regarding detection of the one or more non-invasively detected analytes and the data regarding the detection of the one or more invasively detected analytes, wherein the at least one analyte in common includes the at least one analyte of interest.

18. The method of claim 13, wherein the one or more non-invasively detected analytes and the one or more invasively detected analytes do not include any analytes in common.

19. The method of claim 13, further comprising:
processing the data regarding the detection of the one or more non-invasively detected analytes and the data regarding the detection of the one or more invasively detected analytes to obtain a plurality of assessment factors, and
determining an assessment of a condition of the subject.

20. The method of claim 19, wherein the condition is a response to a treatment provided to the subject.
